(19) United States
(12) Patent Application Publication
(10) Pub. No.: US 2009/0110708 A1
(43) Pub. Date: Apr. 30, 2009

(54) ANIMATE TISSUE ANTISEPSIS

(76) Inventors: Robert C. Platt, Laguna Niguel, CA (US); Szu-Min Lin, Irvine, CA (US); Robert Lukasik, Lake Elsinore, CA (US)

Correspondence Address: K&L GATES LLP, 535 SMITHFIELD STREET, PITTSBURGH, PA, 15222, US

(21) Appl. No.: 11/928,035

(22) Filed: Oct. 30, 2007

(51) Int. Cl. A01N 25/34; A61L 2/22; A01P 1/00

(52) U.S. Cl. 424/405; 422/292

(57) ABSTRACT

A method and device provide for inactivating microorganisms on tissue exposed through a surgical incision. The method includes the steps of: applying a coating of a solution comprising hydrogen peroxide to the tissue; and force drying the solution on the tissue so as to concentrate the solution on the tissue. The concentrated solution kills microorganisms.

### BACKGROUND OF THE INVENTION

The present invention relates to tissue antisepsis, and more specifically to antisepsis of animate tissue, such as before, during, and/or after a surgical procedure.

During a surgical procedure a sterile field is established. The patient is draped with sterile drapes, the skin through which the procedure will be performed is cleaned and disinfected to a high degree, the operating room personnel are garbed in sterile gowns, masks, gloves, shoe covers and the like. Nothing which has not been sterilized, or which may have become contaminated is allowed to enter the sterile field. Even with these precautions it is difficult to prevent any microorganisms from entering the sterile field, such as on dust particles or from the patient. It is desired to have a system and method for addressing contact with open tissue of such microorganisms.

### SUMMARY OF THE INVENTION

A method, according to the present invention, provides for inactivating microorganisms on tissue exposed through a surgical incision. The method includes the steps of: applying a coating of a solution comprising hydrogen peroxide to the tissue; and force drying the solution on the tissue so as to concentrate the solution on the tissue. The concentrated solution kills microorganisms.

Preferably, the step of applying the coating comprises spraying the solution onto the tissue, and more preferably as a fine mist, with an average particle size of less than 5-micrometer diameter, or more preferably less than 2-micrometer diameter.

Preferably, the solution comprises hydrogen peroxide and water with a concentration of hydrogen peroxide in the solution being from about 3% to about 10% by weight, and more preferably from 4% to 8% by weight. In one aspect of the invention, the solution further comprises ethanol in a concentration by weight of from 0.1% to 20%.

In one aspect of the invention, the step of force drying comprises directing a flow of heated air onto the tissue, the heated air preferably having a temperature of from about 30° C. to about 60° C., and more preferably from 45° C. to 55° C. The tissue temperature can optionally be monitored with an infrared temperature sensor.

An apparatus, according to the present invention, provides for inactivating microorganisms on tissue exposed through a surgical incision. The apparatus comprises a source of solution comprising hydrogen peroxide; an applicator for applying the solution to the tissue, the applicator having first exterior surfaces and the first exterior surfaces being sterile; a heat source for applying heat to the tissue to concentrate the solution on the tissue; and the heat source having second exterior surfaces and the second exterior surfaces being sterile.

The first and second exterior surfaces can be separate, or coextensive.

Preferably, the applicator comprises a spray nozzle adapted to provide a mist of the solution with an average particle size of less than 5-micrometer diameter, or more preferably less than 2-micrometer diameter.

Preferably, the solution comprises hydrogen peroxide and water, with the concentration of hydrogen peroxide in the solution being from about 3% to about 10% by weight, and more preferably from 4% to 8% by weight. The solution can also include ethanol in a concentration by weight of from 0.1% to 20%.

Preferably, the heat source is adapted to blow heated air onto the tissue. Also, an infrared temperature sensor can be provided to monitor the tissue temperature.

In one aspect of the invention, the apparatus further comprises a sterilizer comprising a chamber sized to accommodate the applicator, and a source of sterilizing fluid connected to the chamber. Preferably, the source of sterilizing fluid is the source of solution and wherein the sterilizer further comprises a vacuum pump connected to the chamber whereby to lower the pressure within the chamber to vaporize the sterilizing fluid.

### BRIEF DESCRIPTION OF THE DRAWINGS

FIG. 1 is a schematic view of a sterilization device according to the present invention;

FIG. 2 is a graph showing concentration in-situ of a hydrogen peroxide solution; and

FIG. 3 is a graph showing efficacy of the method performed according to the present invention.

### DETAILED DESCRIPTION

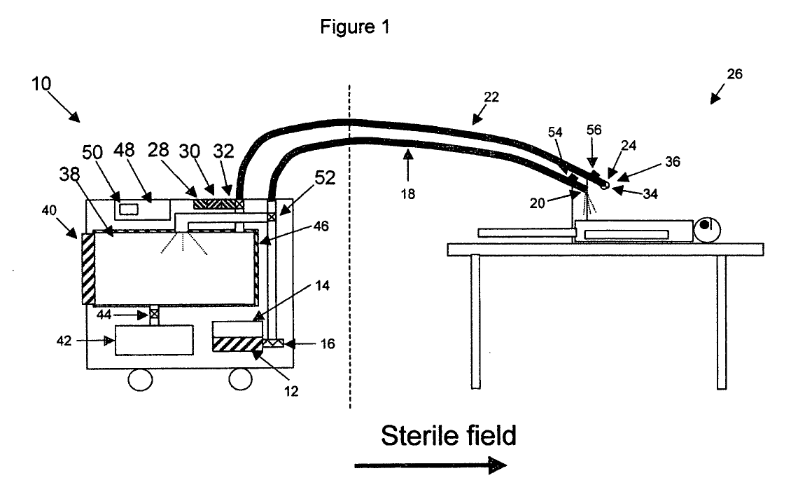

FIG. 1 discloses a sterilization device 10 which can apply a mist of hydrogen peroxide solution 12 comprising water and hydrogen peroxide onto tissue and then apply heat to evaporate the solution 12. In the process of evaporating the water tends to evaporate more quickly than the hydrogen peroxide due to its higher vapor pressure. This causes the remaining solution 12 on the tissue to become ever more concentrated, and this concentration of the solution 12 on the tissue has been shown by the present inventors to effectively inactivate microorganisms thereon.

The sterilization device 10 in gross includes a reservoir 14 of the hydrogen peroxide solution 12. A solution pump 16 carries the solution 12 from the reservoir 14, and in one aspect of the operation directs it toward a supply hose 18 having a spray nozzle 20 for spraying the solution 12 onto tissue. Also, a heater hose 22 having a heated air nozzle 24 supplies heated air to evaporate the solution 12 on the tissue. The heated air causes the solution 12 to evaporate and concentrate the hydrogen peroxide on the tissue. Each of the supply hose 18 and spray nozzle 20 and the heater hose 22 and air nozzle 24 are sterile so that they can enter a sterile field 26.

To supply the heated air the device 10 includes a fan 28 which draws in air through an inlet filter 30 which filters out potentially contaminating microorganisms and then passes the air over a heating element 32, preferably a simple electric resistance element, and then on to the heater hose 22. Alternatively, or cumulatively, an infrared (IR) heating element 34 can be provided at a distal end 36 of the heater hose 22. Further, a temperature sensor, such as an IR sensor (not shown) can be provided to monitor temperature of the tissue to provide optimum evaporation without overheating the tissue.

Preferably, the hydrogen peroxide solution 12 comprise approximately four to ten percent hydrogen peroxide with the remainder being mostly water with perhaps other ingredients such as stabilizers to prevent breakdown of the hydrogen peroxide etc. More concentrated solutions provide potential benefits in efficacy, which must be matched against the potential to irritate the tissue to which they are applied. The optimal pH at which hydrogen peroxide attacks microorganisms is below neutral (less than 6) and it may be beneficial to add pH lowering agents such as acetic acid etc. which are friendly to animate tissue. Again, this must be balanced against the irritation that a lower than body pH may cause to the tissue. Alcohol, preferably simple alcohols such as ethyl alcohol, can also be added, or even substituted for the water. The alcohol has the added advantage of evaporating even more quickly than water and also is a potent disinfectant.

The sterilization device 10 with the so far mentioned basic features could be provided in a very compact form factor, especially if the pump 16 were manually operated, and heat were provided by the IR element 34 powered by a battery (not shown). Even a simple battery operated pump could be used for the distribution pump 16 to keep the device 10 small enough to be handheld.

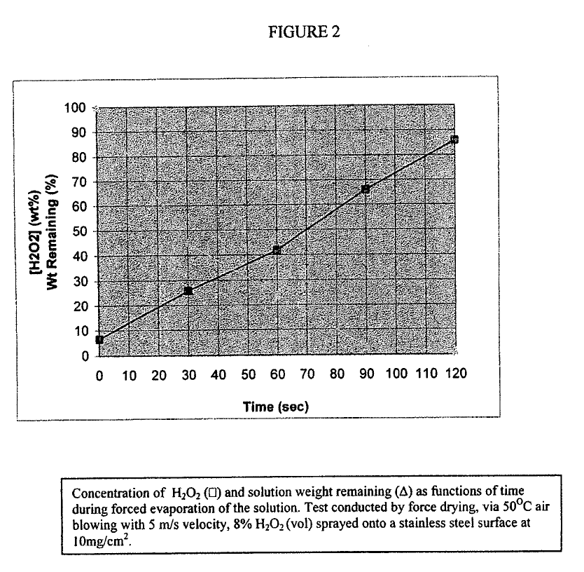

Antisepsis of tissue is accomplished by enhanced evaporation of the hydrogen peroxide solution 12 applied to the tissue surface. Providing the hydrogen peroxide is in solution with compounds that have higher vapor pressure than itself, e.g., water or alcohol, forced evaporation will rapidly concentrate the hydrogen peroxide to bactericidal levels so achieving antisepsis of the treated surface. Here “forced evaporation” is evaporation a rate faster than would be normal for the solution at ambient conditions under which it is applied. It may be accomplished by, e.g., flowing heated or unheated air over the treated surface, heating of the surface itself by IR radiation, heating of the solution after application by chemical reaction or reducing local pressure (applying vacuum). Concentration of peroxide during forced evaporation is shown in FIG. 2.

Forced evaporation is preferred for treatment of living tissues because these tissues are typically continually moist and so will not dry and concentrate the H₂O₂ solutions without it. For instance, the peritoneal cavity (one tissue surface particularly targeted for this technique) is lined by the peritoneum, a serous membrane that has transudate (peritoneal fluid) formed on its surface continuously. Evaporation must concentrate the peroxide solution combined with the transudate in the treated area thus forced evaporation is much preferred.

Furthermore, forced evaporation helps clear the H₂O₂ from the treatment site as well, thus minimizing the amount remaining after treatment. This is not a rigorous requirement, however, as an important advantage to H₂O₂ is that it is tolerated by the body tissues in that agents (e.g., catalase) to chemically eliminate oxidizers such as peroxide are manufactured by the body and present throughout its tissue.

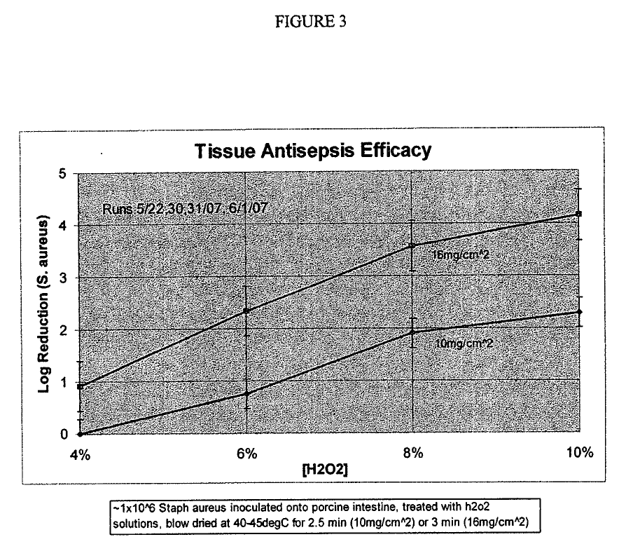

Staphylococcus aureus inoculated onto inanimate porcine intestine can be significantly killed by enhanced evaporation of H₂O₂ with initial concentrations of 4%-10% (vol.). FIG. 3 shows death of the organisms to be an approximately log-linear function of initial peroxide concentration. These results were obtained by evaporating the H₂O₂ solution 12 (applied to the tissue at 16 mg/cm²) via blowing air (temperature 50° C., velocity 5.0 m/sec at the tissue surface) at the treated tissue.

Addition of alcohol or other high volatility compounds to the solution can reduce the evaporation time. Data shows that the same log reduction can be achieved with 10% (vol) alcohol with 2.5 minutes drying time compared to 3 minutes drying without alcohol. Alcohol concentration should be kept below approximately 20% to avoid fire hazard, intoxication of operating room personnel and to ensure complete evaporation of the alcohol before the surgical wound is closed.

Although alcohol offers benefit in terms of reduced drying times, its addition is not strictly necessary. Results of testing with no alcohol show log reductions of 2.21 (3 minute dry time), 2.68 (3.5 minute dry time) and 1.79 (4 minute dry time).

The measured reductions in bacterial titer is due almost entirely to H₂O₂: experiments using solutions containing no peroxide and 10% alcohol (EtOH) showed no measurable log reduction.

One concern is sterilization of the supply hose 18 and heater hose 22. As they enter the sterile field 26 they must be sterile. They can be sterilized in a sterilizer in the hospital, or be provided sterile in sterile packaging from a manufacturer. The device 10 contains basic components which might form a sterilizer and can in one aspect of the invention comprise a sterilization chamber 38.

The chamber 38 includes an access door 40 and has room to accommodate at least the supply hose 18 and heater hose 22, which are detachable from the device 10. Preferably a vacuum pump 42 fluidly connects to the chamber 38 through a valve 44. A heating element 46 provides heat to the chamber 38. A control computer 48 and input/output screen 50 control operation of the device 10 and provide user input.

To sterilize the supply hose 18 and heater hose 22, they are detached and placed into the chamber 38 through the door 40. The chamber 38 is sealed and a vacuum is drawn upon the chamber 38 via the vacuum pump 42. In some commercial hydrogen peroxide sterilizers the vacuum reaches 0.5 torr, but that requires an expensive vacuum pump 42. A vacuum below about 200 torr provides an effective compromise. Hydrogen peroxide solution 12 is then pumped into the chamber 38 (via control of a valve 52) and preferably supplied as a mist. Alternatively, a heated vaporizer (not shown) can vaporize the solution into a gas. Preferably, the chamber 38 is heated to about 50° C. prior to admitting the peroxide solution 12. The peroxide solution for sterilization may be different than the peroxide solution for treating tissue, and preferably comprises a higher concentration of hydrogen peroxide.

The peroxide is allowed time to diffuse into lumens and time for contact with surfaces and effect sterilization thereof. The lumens may be pre-treated with liquid peroxide before placing into the chamber 38. Preferably, a cycle for sterilization involves a vacuum of 0.1 to 200 torr, a temperature of 18 to 50° C., for 5 to 30 minutes, with a 4 to 60% hydrogen peroxide solution at a concentration of 0.2 to 20 mg/liter of chamber volume. Fresh air is then admitted through the filter 30. To reduce residual peroxide, another vacuum and vent series can be performed on the chamber 38, with the temperature of 30 to 50° C. maintained by the heating element 32. The spray hose 18 and heater hose 22 are then sterile and ready for use in tissue antisepsis.

For tissue antisepsis, after opening an incision into a patient, or for other antisepsis of exposed tissue, a spray of hydrogen peroxide solution is provided through the spray nozzle 20 to such tissue. Preferably, a spray button 54 is provided adjacent the spray nozzle 20, or alternatively a foot switch (not shown) could be provided on the device 10. Then, heat is applied from the heater hose 22 and nozzle 24 to evaporate and concentrate the solution 12 on the tissue. Again, a heat switch 56 is preferably provided at the nozzle 24. This process can be repeated during the course of a surgical procedure or even be operated continuously during such to maintain sterility to a high degree on the exposed internal tissue. This antisepsis treatment can also be applied to the skin of the patient to prepare the patient surgical site before the surgery.

The sterilization chamber 38 can also be used for flash sterilization of an instrument in the operating theater during a surgical procedure. This can be particularly useful if a critical instrument is contaminated during a procedure. The instrument is placed into the chamber and a sterilization cycle such as used on the supply hose 18 and heater hose 22 can be performed. The cycle can also be different, such as if the instrument has no lumens etc. to be faster.

The device 10 is shown with a separate supply hose 18 and heater hose 20 yet these could be combined into a single hose with separate lumens for the peroxide solution and heater air. Also, a separate source of peroxide (not shown) having a much higher concentration peroxide, such as 59% which is currently the highest concentration conveniently shippable, can be provided for sterilization cycles within the chamber 38 with the lower, more tissue friendly concentration being in the reservoir 14. The reservoir 14 could also have the higher concentration with a diluting fluid of water or other solvent being provided for the tissue antisepsis portion of the method. Variations on the cycle in the chamber 38 are possible, and optimum cycles therein are discussed in U.S. Pat. Nos. 5,851,485, 5,804,139, 5,980,825, 6,451,254, 6,528,017, 6,656,426, and 6,673,313, 6,977,061 the contents of which are incorporated herein by reference.

The invention has been described with reference to the preferred embodiments. It is intended that the invention be construed as including all modifications and alterations insofar as they come within the scope of the appended claims or the equivalents thereof.

What is claimed is:

1. A method for inactivating microorganisms on tissue exposed through a surgical incision, the method comprising the steps of:
applying a coating of a solution comprising hydrogen peroxide to the tissue; and
force drying the solution on the tissue so as to concentrate the solution on the tissue.

2. A method according to claim 1 wherein the step of applying the coating comprises spraying the solution onto the tissue.

3. A method according to claim 2 wherein the solution is sprayed as a fine mist.

4. A method according to claim 3 wherein the mist has an average particle size of less than 5-micrometer diameter.

5. A method according to claim 4 wherein the mist has an average particle size of less than 2-micrometer diameter.

6. A method according to claim 1 wherein the solution comprises hydrogen peroxide and water and wherein a concentration of hydrogen peroxide in the solution is from about 3% to about 10% by weight.

7. A method according to claim 6 wherein the concentration of hydrogen peroxide in the solution is from 4% to 8% by weight.

8. A method according to claim 1 wherein the solution further comprises ethanol in a concentration by weight of from 0.1% to 20%.

9. A method according to claim 1 wherein the step of force drying comprises directing a flow of heated air onto the tissue.

10. A method according to claim 9 wherein the heated air has a temperature of from about 30° C. to about 60° C.

11. A method according to claim 10 wherein the heated air has a temperature of from 45° C. to 55° C.

12. A method according to claim 1 and further comprising monitoring the tissue temperature with an infrared temperature sensor.

13. An apparatus for inactivating microorganisms on tissue exposed through a surgical incision, the apparatus comprising:
a source of solution comprising hydrogen peroxide;
an applicator for applying the solution to the tissue, the applicator having first exterior surfaces and the first exterior surfaces being sterile;
a heat source for applying heat to the tissue to concentrate the solution on the tissue; and
the heat source having second exterior surfaces and the second exterior surfaces being sterile.

14. An apparatus according to claim 13 wherein the first exterior surfaces are coextensive with the second exterior surfaces.

15. An apparatus according to claim 13 wherein the applicator comprises a spray nozzle.

16. An apparatus according to claim 15 wherein the spray nozzle is adapted to provide a mist of the solution and wherein the mist has an average particle size of less than 5-micrometer diameter.

17. An apparatus according to claim 16 wherein the spray nozzle is adapted to provide a mist of the solution and wherein the mist has an average particle size of less than 2-micrometer diameter.

18. An apparatus according to claim 13 wherein the solution comprises hydrogen peroxide and water and wherein the concentration of hydrogen peroxide in the solution is from about 3% to about 10% by weight.

19. An apparatus according to claim 14 wherein the concentration of hydrogen peroxide in the solution is from 4% to 8% by weight.

20. An apparatus according to claim 18 wherein the solution further comprises ethanol in a concentration by weight of from 0.1% to 20%.

21. An apparatus according to claim 13 wherein the heat source is adapted to blow heated air onto the tissue.

22. An apparatus according to claim 13 and further comprising an infrared temperature sensor to monitor the tissue temperature.

23. An apparatus according to claim 13 and further comprising a sterilizer comprising a chamber sized to accommodate the applicator, and a source of sterilizing fluid connected to the chamber.

24. An apparatus according to claim 23 wherein the source of sterilizing fluid is the source of solution and wherein the sterilizer further comprises a vacuum pump connected to the chamber whereby to lower the pressure within the chamber to vaporize the sterilizing fluid.
